(19) United States
(12) Patent Application Publication
(10) Pub. No.: US 2002/0166555 A1
(43) Pub. Date: Nov. 14, 2002

(54) Underwater breathing apparatus

(76) Inventor: Steven F. Antonelli, Revere, MA (US)

Correspondence Address: STEVEN F. ANTONELLI, 28 ELMWIID AVENUE, REVERE, MA, 02151, US

(21) Appl. No.: 09/853,175

(22) Filed: May 11, 2001

(51) Int. Cl. B63C 11/16; B63C 11/02; A62B 7/00; A61M 15/00

(52) U.S. Cl. 128/201.11; 128/201.27; 128/202.14

(57) ABSTRACT

An apparatus for breathing under water wherein a direct air link is established between a diver and the surface air. The apparatus is comprised of mouthpiece member coupled to the first tube where the first tube is coupled to a container. The container is secured next to a diver's body by means of a harness. The container has a second tube extending from it to the water surface. At the surface, the second tube is coupled to a floating device. The floating device has a passage for allowing air in and out of the second tube. In a preferred embodiment, the mouthpiece member is equipped with a check valve. The check valve allows the air from the first tube to be inhaled by the diver while discharging exhaled air into the water.

### FIELD OF THE INVENTION

This invention relates to an apparatus that allows a diver to breathe surface air while submerged under water and to a method of utilizing this apparatus for breathing under water.

Man has attempted to explore underwater realms for centuries. A number of different solutions were found. One is to use a snorkel mechanism. However, with a snorkel, a diver must remain at the surface of the water and if he wishes to dive deeper he must hold his breath until he comes up to the surface. An additional problem with a snorkel is that the water can easily get into the snorkel and get inhaled. Another method of diving is to use such well known mechanisms as SCUBA and its derivatives. However, the drawback of SCUBA is that it limits a diver's underwater time to the amount of air stored in a tank as well as involving wearing of complicated, heavy, and bulky equipment.

What is needed is a simple, easy to use device that would allow a diver to remain under water for an indefinite amount of time without a danger of water entering into diver's mouth and lungs.

### SUMMARY OF THE INVENTION

This invention satisfies the above needs. A novel apparatus for breathing under water is provided.

The claimed apparatus has a hollow container, a harness with which a diver could mount the container on his back, a first tube connecting the container to a mouthpiece member, and a second tube which runs from the container to a floating device. The mouthpiece member is located at a middle section of the first tube. The diver holds the mouthpiece member in his mouth. The floating device remains buoyant on the surface of the water and it has a passage for allowing air in and out of the second tube. When the diver is submerged, he inhales air through the mouthpiece member. As he does so, the air is sucked from the air above the surface of the water, into the passage inside of the floating device, then into the second tube, passing along into the container, the first tube, and finally into the mouthpiece device. Thus, there exists an uninterrupted air link from the area above the surface of the water and into the mouth of the diver. Furthermore, the floating device, by remaining buoyant on the surface, prevents water from getting into the air link. For exhaling, the diver could either simply exhale into the mouthpiece member and thus let the exhaled air move backwards through the air link described above, or if, as in one of the embodiments of the invention, there is a check valve present in the mouthpiece member, then the exhaled air would go straight from the mouthpiece member into the surrounding water. Since, there is no limitation on the air supply, the diver could remain submerged for an indefinite amount of time.

### BRIEF DESCRIPTION OF DRAWINGS

These and other features, aspects and advantages of the present invention will become better understood with reference to the following description, appended claims, and accompanying drawings where

FIG. 1 is a depiction of one of the embodiments of the underwater breathing apparatus in use by a diver;

FIG. 2 is a depiction of another embodiment of the underwater breathing apparatus in use by a diver;

FIG. 3 is a side view of one of the embodiments of the invention;

FIG. 4 is a side view of another embodiment of the invention;

FIG. 5 is a side view of one of the embodiments of the container of the underwater breathing apparatus.

### DESCRIPTION OF THE INVENTION

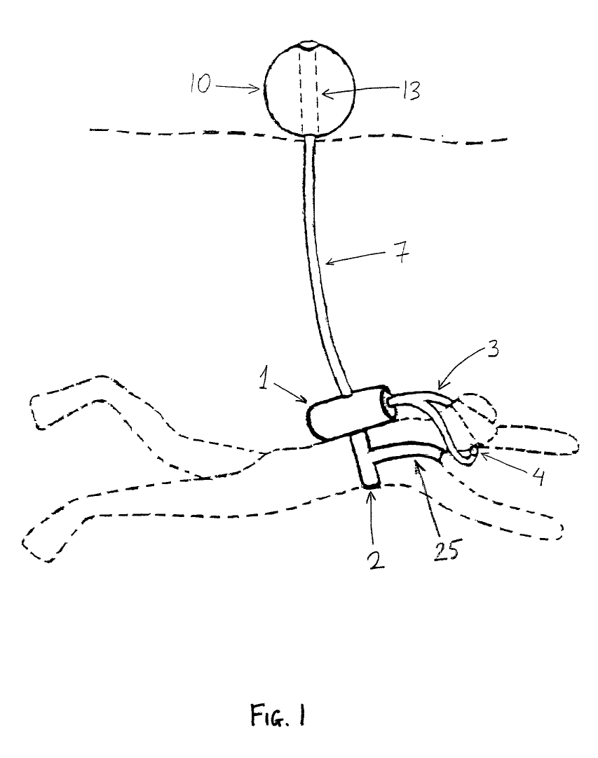

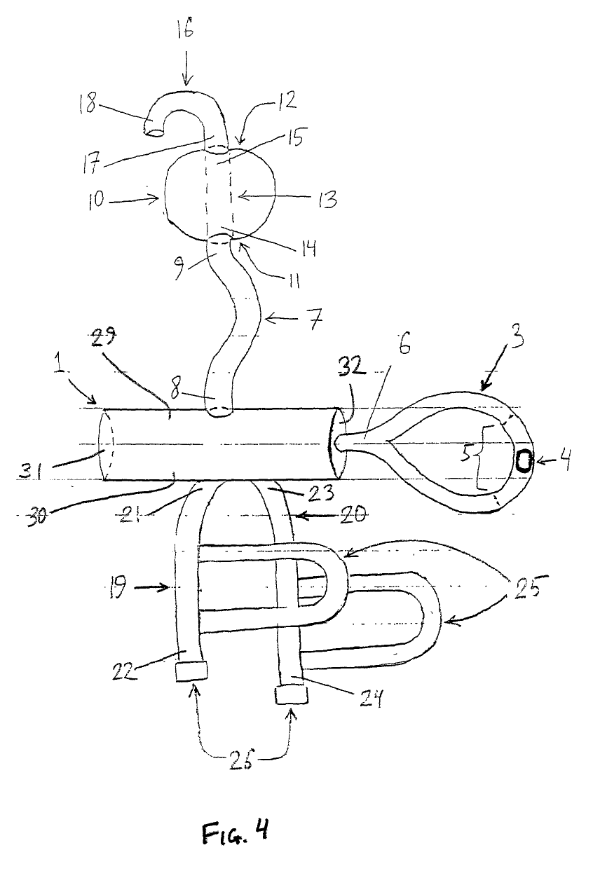

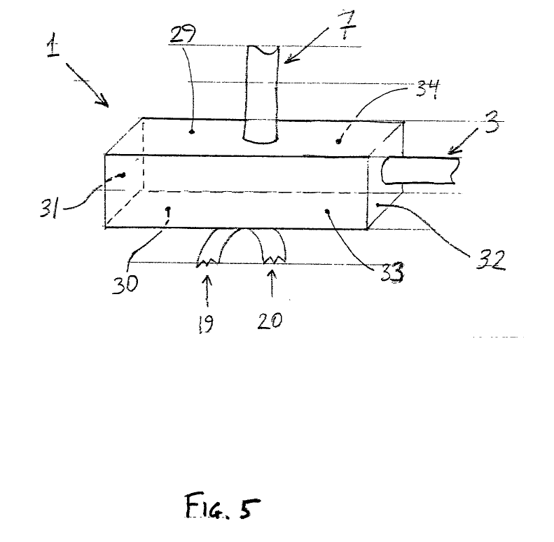

This invention represents an apparatus and a method for breathing under water. It is comprised of several interconnected parts where all parts are made out of airtight, non-corrosive material. A hollow container 1 is secured onto the back of a diver by means of a harness 2 as depicted in FIG. 1. The container 1 could have a plurality of shapes. When the container 1 has a barrel shape it has a front portion 32, a back portion 31, a top portion 29, and a lower portion 30 as depicted in FIG. 4. When the container 1 has a cubical shape it also additionally has a left portion 34 and a right portion 33 as depicted in FIG. 5.

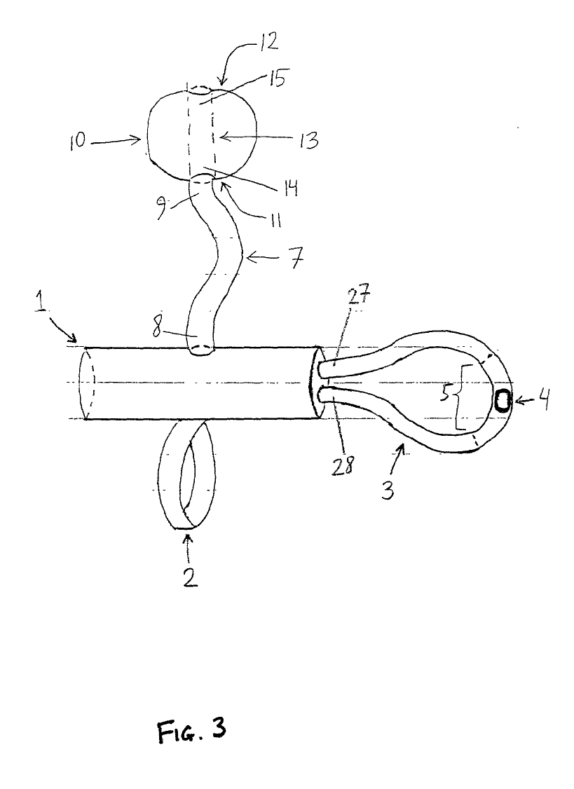

The harness 2 could be a circular single piece elastic strip as the one depicted in FIG. 3. The harness 2 could also be made out of two strips 19 and 20, where the first strip 19 has a first end 21 and a second end 22 and the second strip 20 has a first end 23 and a second end 24. The two strips, 19 and 20, are coupled by their respective first ends, 21 and 23 to the container 1. The two strips, 19 and 20, are equipped with a lacking mechanism 26 at their respective second ends 22 and 24. By utilizing the locking mechanism 26, the two strips, 19 and 20, could engage each other thus securing the apparatus to the diver. The harness 2 could also have shoulder straps 25, coupled to the harness strips 19 and 20 as depicted in FIGS. 1, 2, and 4.

The diver would secure a mouthpiece member 4 onto his mouth. The mouthpiece member 4 is located in the middle section 5 of a first tube 3. The first tube 3 could have a first end 27 and a second end 28, which ends couple to the container 1 in the front portion 32 of the container 1 as depicted in FIG. 3. The first tube 3 could also have its ends 27 and 28 couple together to form a unified end 6 which end would then couple to the front portion 32 of the container 1 as depicted in FIG. 4.

The second tube 7 has a first end 8 and a second end 9 as depicted in FIGS. 3 and 4. The first end 8 is coupled to the top portion 29 of the container 1 as depicted in FIGS. 1, 3, 4, and 5.

The floating device 10 has a subsurface end 11 and a surface end 12 as depicted in FIGS. 3 and 4. The floating device 10 also has going through it a passage 13 which passage has a first end 14 and a second end 15 as depicted in FIGS. 3 and 4. The first end 14 of the passage 13 is located at the subsurface end 11 of the floating device 10 while the second end 15 of the passage 13 is located at the surface end 12 of the floating device 10 as depicted in FIGS. 3 and 4.

The second end 9 of the second tube 7 is coupled to the subsurface end 11 of the floating device 10 as depicted in FIGS. 3 and 4.

### DESCRIPTION OF THE PREFERRED EMBODIMENTS

In one preferred embodiment of the invention, the mouthpiece member 4 has a check valve built into it. The check valve is of a kind that is commonly known in the art. The check valve allows the inhaled air to pass from the first tube into the mouthpiece member while discharging the exhaled air from the mouthpiece member into the surrounding water.

In another preferred embodiment, the surface end 12 of the floating device 10 is coupled to a first end 17 of a curved hollow tube 16 as depicted in FIG. 4. The second end 18 of the curved tube 16 is open to the ambient atmospheric air as depicted in FIG. 4. The curved tube becomes an extra link between the passage inside of the floating device and the air above the water surface. The advantage of having the curved tube 16 is that, in case of rain, it will prevent rainwater from entering into the air link. Another advantage of the curved tube 16 is that, in case of choppy seas, it will prevent water from splashing waves from getting into the air link.

In another preferred embodiment, all the parts of the apparatus are made out of a light, airtight, non-corrosive material. The container 1 is made out of such material as plastic, the first tube 3 and the second tube 7 are made out of such material as bendable plastic or rubber, the floating device 10 is made out of such water surface buoyant material as plastic or wood, and the curved tube 16 could be made out of such material as rubber or plastic.

In another preferred embodiment, the container 1 has a barrel shape as depicted in FIGS. 1, 3, and 4 with a length of 0.35 meters, diameter of 0.17 meters, and a thickness of 0.005 meters. The first tube 3, the second tube 7, and the curved tube 16, all have a cylindrical shape with an outer diameter of 0.07 meters and an inner diameter of 0.065 meters. The first tube 3 has a length of 2.0 meters. The second tube 7 has a length of 14.0 meters. The floating device 10 has a spherical shape with a diameter of 0.35 meters, and the passage 13 has a cylindrical shape and a diameter of 0.065 meters.

In another preferred embodiment the second end 9 of the second tube 7 passes into the passage 13 at the subsurface end 11 of the floating device 10 and comes out at the surface end 12 of the floating device 10 through the second end 15 of the passage 13 so that the portion of the second tube 7 that is extended above the floating device 10 becomes the equivalent of the curved tube 16 in other embodiments.

In another preferred embodiment, the curved tube 16 has an L shape where the second end 18 of the curved tube 16 is pointing in a horizontal direction. The curved tube 16 could also have a U shape where the second end 18 of the curved tube 16 is pointing downwards as depicted in FIG. 4.

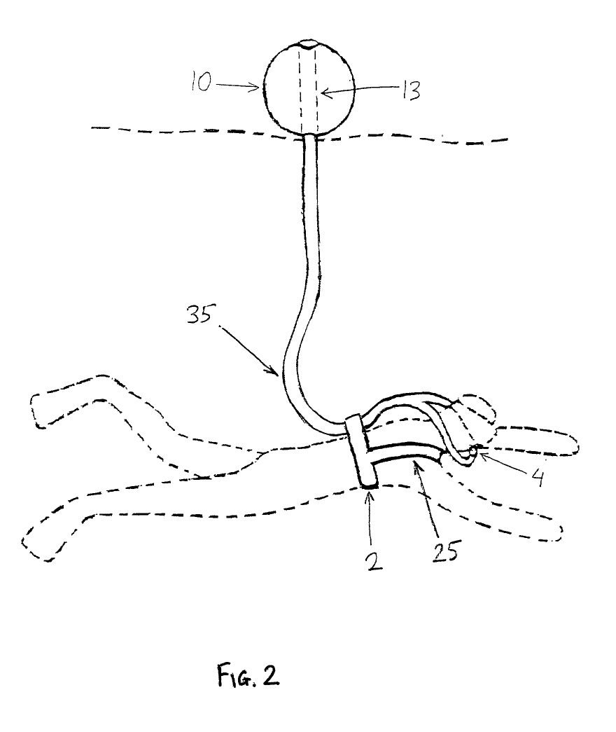

In another preferred embodiment, the container 1 is absent and the unified end 6 of the first tube 3 is coupled directly to the first end 8 of the second tube 7 forming a singular connection tube 35 as depicted in FIG. 2. In this embodiment the atmospheric air will pass from through the passage 13, into the connection tube 35 and into the mouthpiece member 4. Here the harness 2 is coupled directly to the connection tube 35 as depicted in FIG. 2.

What I claim is:

1. Apparatus for breathing under water, which comprises:
an airtight hollow container;
a harness coupled to the container;
an airtight mouthpiece member;
first airtight hollow tube having first and second ends and a middle section wherein the middle section is coupled to the mouthpiece member and the first and the second ends are coupled to the container and wherein the cavity inside of the first tube is in fluid communication with a cavity inside of the container;
a second airtight hollow tube having first and second ends wherein the first end of the second tube is coupled to the container wherein a cavity inside of the second hollow tube is in fluid communication with the cavity inside of the container.
a floating device capable of floating on a water surface, having surface and subsurface ends and a hollow passage passing throughout the floating device with first end of the passage located at the subsurface end of the floating device and a second end of the passage located at the surface end of the floating device;
the second end of the second tube is coupled with the subsurface end of the floating device wherein the cavity inside of the second tube is in fluid communication with the passage inside of the floating device.

2. Apparatus for breathing under water of claim 1, wherein the mouthpiece member has a check valve wherein the check valve is capable of allowing inhaled air to pass from the first tube into the mouthpiece member and capable of discharging exhaled air into the water;

3. Apparatus for breathing under water of claim 1, wherein the first tube has a length of 0.50 to 4.00 meters and the second tube has a length of 0.10 to 20.00 meters.

4. Apparatus for breathing under water of claim 1, wherein the first end of the first tube is coupled with the second end of the first tube wherein a cavity inside of the first end of the first tube is in fluid communication with the cavity inside of the second end of the first tube, thereby creating a singular unified end of the first tube, where the unified end is coupled to the container and wherein the cavity inside of the first tube is in fluid communication with the cavity inside of the container.

5. Apparatus for breathing under water of claim 1, wherein the floating device by remaining buoyant on the surface of the water prevents water from entering the second end of the passage.

6. Apparatus for breathing under water of claim 1, wherein the floating device has a spherical shape with an outer diameter of 0.10 to 1.00 meters and where the passage inside of the floating device has a cylindrical shape with a diameter of 0.003 to 0.50 meters.

7. Apparatus for breathing under water of claim 1, wherein the floating device has a singular or a plurality of inner cavities filled with air and where the floating device is capable of remaining buoyant on water surface.

8. Apparatus for breathing under water of claim 1, wherein the floating device is made out of a buoyant material which allows the floating device to remain buoyant on water surface.

9. Apparatus for breathing under water of claim 1, further comprising:
a curved hollow tube which has a first and second ends wherein the first end of the curved tube is coupled to the surface end of the floating device and wherein the cavity inside of the curved tube is in fluid communication with the passage and where the second end of the curved tube is open to the ambient air above the water surface.

10. Apparatus for breathing under water of claim 1, wherein the floating device by remaining buoyant on the surface of the water prevents water from entering the second end of the curved tube.

11. Apparatus for breathing under water of claim 1, wherein the container has a barrel shape with an outer diameter of 0.05 to 1.00 meters, an inner diameter of 0.048 to 0.998 meters, a lower, upper, front, and back portions where the lower portion mounts next to a diver when the apparatus of claim 1 is secured onto a diver by means of the harness, the upper portion is coupled to the first end of the second tube and the front portion is coupled to the first tube.

12. Apparatus for breathing under water of claim 1, wherein the container has a rectangular cubical shape with a length of 0.01 to 1.00 meters, a width of 0.01 to 1.00 meters, and a height of 0.01 to 1.50 meters, with walls of the container having a thickness of 0.002 to 0.100 meters, and having a lower, upper, front, back, left and right portions and where the lower portion mounts next to a diver when the apparatus of claim 1 is secured onto a diver by means of the harness, the upper portion is coupled to the first end of the second tube and the front portion is coupled to the first tube.

13. Apparatus for breathing under water of claim 1, wherein the container has a spherical shape with an outer diameter of 0.02 to 1.50 meters and an inner diameter of 0.018 to 1.498 meters.

14. Apparatus for breathing under water of claim 1, wherein the container is absent and the unified end of the first tube is coupled to the first end of the second tube wherein the cavity inside of the first tube is in fluid communication with the cavity inside of the second tube whereby the first and the second tubes form a singular connection tube and wherein the harness is coupled to the connection tube.

15. Apparatus for breathing under water of claim 1, wherein the first tube is made out of a flexible material with an outer diameter of 0.005 to 0.20 meters and an inner diameter of 0.003 to 0.198 meters.

16. Apparatus for breathing under water of claim 1, wherein the second tube is made out of a flexible material with an outer diameter of 0.005 to 0.20 meters and an inner diameter of 0.003 to 0.198 meters.

17. Apparatus for breathing under water of claim 1, wherein the harness is made out of an elastic material.

18. Apparatus for breathing under water of claim 1, wherein the harness further comprises:
a first strip and a second strip wherein the first strip has a first and second ends where the first end if the first strip is affixed to the container and where the second strip has a first and second ends where the first end of the second strip is affixed to the container and where the second end of the first strip is capable of engaging with the second end of the second strip, wherein second ends of the first and second harness strips are coupled to a locking mechanism capable of engaging and disengaging the second ends of the first and second harness strips, wherein the first harness strip is coupled with a shoulder strap and the second harness strip is coupled with a shoulder strap.

19. A method of breathing under water, which comprises:
securing of the apparatus described in claim 1 to a human body by way of the harness;
inhaling through use of the apparatus described in claim 1 wherein air passes from the air above the surface of the water into the passage of the floating device, into the second tube, into the container, into the first tube, into the mouthpiece member, and then into the diver's mouth and lungs;
exhaling by through use of the apparatus described in claim 1 wherein exhaled air passes from the mouth of the diver into the mouthpiece member, into the first tube, into the container, into the second tube, into the passage of the floating device, and is released into the air above the surface of the water.

20. A method of breathing under water, which comprises:
securing of the apparatus described in claim 2 to a human body by way of the harness;
inhaling by through use of the apparatus described in claim 2 wherein air passes from the air above the surface of the water into the passage of the floating device, into the second tube, into the container, into the first tube, into the mouthpiece member through the check valve, and then into the diver's mouth and lungs;
exhaling through use of the apparatus described in claim 2 wherein exhaled air passes from the mouth of the diver into the mouthpiece member, and is released into the water through the check valve.
